(19) United States
(12) Patent Application Publication
(10) Pub. No.: US 2017/0135892 A1
(43) Pub. Date: May 18, 2017

(54) Trigger Point Treatment Device

(71) Applicant: Tech Tank, LLC, Erie, PA (US)

(72) Inventors: Lucas McConnell, Erie, PA (US); Brock Allen, Erie, PA (US)

(73) Assignee: Tech Tank, LLC, Erie, PA (US)

(21) Appl. No.: 15/350,580

(22) Filed: Nov. 14, 2016

Related U.S. Application Data
(60) Provisional application No. 62/254,535, filed on Nov. 12, 2015.

(51) Int. Cl. A61H 1/00; A61N 1/36

(52) U.S. Cl. CPC A61H 1/008; A61N 1/36021; A61H 2201/0207; A61H 2201/1261

(57) ABSTRACT

A trigger point treatment device comprises a convex side that has a working area against which a user may present trigger point areas for the application of self-applied pressure. A concave side on the other side of the trigger point treatment device is used for mounting the trigger point treatment device to a surface by orienting the concave side against the surface and applying pressure against the convex side to create negative pressure between said concave side and the surface.

### BACKGROUND

Myofascial trigger points, also known as trigger points, are described as spots in the soft tissue, the fascia, of the human body surrounding skeletal muscle. Trigger points are often associated with knots or myofascial pain syndrome. Applying pressure to or massaging these trigger points is known to provide at least temporary relief from pain associated with the trigger points. What is presented is a portable trigger point treatment device that is positionable on vertical or horizontal surfaces and allows the user to provide self-applied pressure against the device to massage their own trigger points.

### SUMMARY

What is presented is a trigger point treatment device comprising a convex side that has a working area against which a user may present trigger point areas for the application of self-applied pressure. A concave side on the other side of the trigger point treatment device is used for mounting the trigger point treatment device to a surface by orienting the concave side against the surface and applying pressure against the convex side to create negative pressure between the concave side and the surface. The working area of the trigger point treatment device is used for any of the temporary relief of myofascial pain syndrome, massage therapy, minor pain relief, muscle soreness, and muscle fatigue. The trigger point treatment could be made from plastic, rubber, or other suitable material.

The working area of the trigger point treatment device may be modified to have a heat generation device, an electrical pulse generation device, or a vibration generation device to supplement the self-applied pressure that a user places against the trigger point treatment device. The working area of the trigger point treatment device could comprise a single shape protruding towards the user or multiple shapes protruding towards the user. In various embodiments the working area could have a shape that is one of conical, spheroidal, orbicular, ball shaped, or peaked. In addition, in other embodiments, the working area could be detachable from the convex side.

Those skilled in the art will realize that this invention is capable of embodiments that are different from those shown and that details of the apparatus and methods can be changed in various manners without departing from the scope of this invention. Accordingly, the drawings and descriptions are to be regarded as including such equivalent embodiments as do not depart from the spirit and scope of this invention.

### BRIEF DESCRIPTION OF DRAWINGS

For a more complete understanding and appreciation of this invention, and its many advantages, reference will be made to the following detailed description taken in conjunction with the accompanying drawings.

FIG. 1 is a perspective top view of an embodiment of trigger point treatment device;

FIG. 2 is a perspective bottom view of the trigger point treatment device of FIG. 1;

FIG. 3 shows the trigger point treatment device of FIG. 1 mounted to a surface and illustrates its use;

FIG. 4 is a perspective bottom view of another embodiment of trigger point treatment device;

FIG. 5 is a perspective bottom view of another embodiment of trigger point treatment device;

FIG. 6 is a side view of another embodiment of trigger point treatment device;

FIG. 7 is a side view of another embodiment of trigger point treatment device;

FIG. 8 is a perspective view of another embodiment of trigger point treatment device; and

FIG. 9 is a perspective view of another embodiment of trigger point treatment device.

### DETAILED DESCRIPTION

Referring to the drawings, some of the reference numerals are used to designate the same or corresponding parts through several of the embodiments and figures shown and described. Corresponding parts are denoted in different embodiments with the addition of lowercase letters. Variations of corresponding parts in form or function that are depicted in the figures are described. It will be understood that variations in the embodiments can generally be interchanged without deviating from the invention.

Back pain and muscle soreness are commonplace ailments of most adults. Going to a massage therapist is not always an option. What is presented is a trigger point treatment device that is small, portable, and able to be positioned where a user may be able to make best use of self-applied pressure against myofascial trigger points (“trigger points”) for relief of pain and muscle soreness. The trigger point treatment device is positionable on vertical or horizontal surfaces and allows the user to provide self-applied pressure against the device to massage their own trigger points.

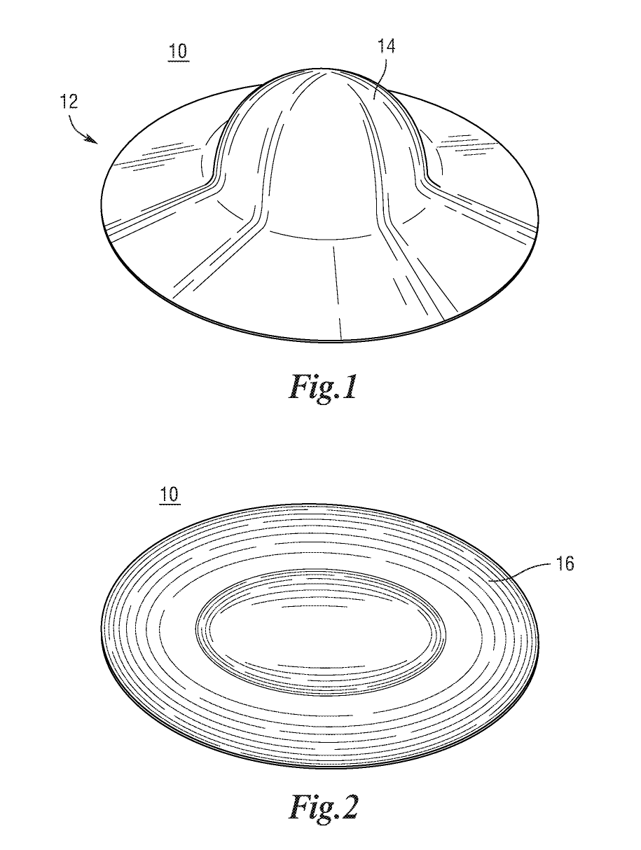

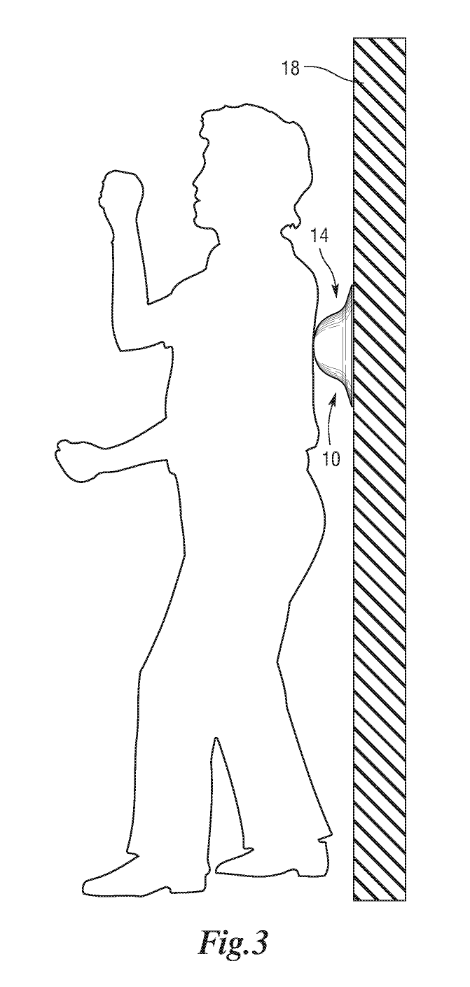

As best understood by comparing FIGS. 1, 2, and 3, shows a trigger point treatment device 10 that comprises a convex side 12 that has a working area 14 against which a user may present trigger point areas for the application of self-applied pressure. Opposite the convex side 12 is a concave side 16 for mounting the trigger point treatment device 10 to a surface 18. This is done by orienting the concave side 16 against the surface 18 and applying pressure against the convex side 12 to create negative pressure between the concave side 16 and the surface 18. With the trigger point treatment device 10 positioned on the surface 18, a user may present those trigger points against the working area 14 that require temporary relief of myofascial pain syndrome, massage therapy, minor pain relief, muscle soreness, and muscle fatigue. The surface 18 may be a wall as shown in FIG. 3, but it could also be a floor for a user to be able to stand on the working area 14 to treat their feet. In fact any flat surface against which the trigger point treatment device 10 may adhere would suffice if it allows the working area 14 to be accessible to the user as needed.

The trigger point treatment device 10 may be made from any suitable material that allows the convex side 12 to create negative pressure between it and the surface 20. Some suitable materials include elastomeric materials such as rubber and some plastics. The trigger point treatment device 10 could be a single piece as shown in FIGS. 1 through 3 or it could comprise multiple components as shown and discussed for example later herein.

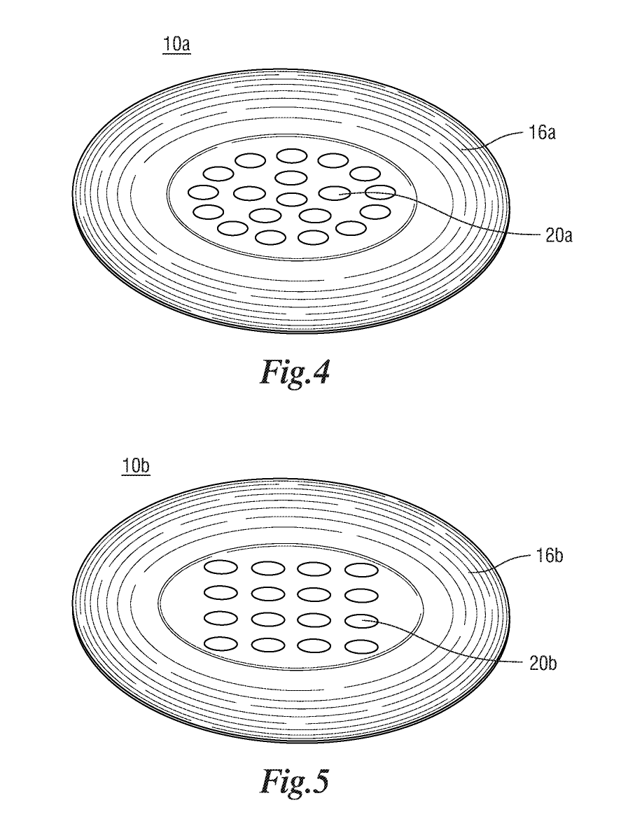

FIG. 2 shows that the convex side 16 of the trigger point treatment device 10 to be a smooth surface, but FIG. 4 shows an embodiment in which the surface has material removed to form openings 20a that extend into the working area. This changes the stiffness of the working area and allows for embodiments in which trigger point treatment devices 10a may be stiffer or softer depending on the number and arrangement of openings 20a to allow for the personal preference of the user. This also has the effect of making the trigger point treatment device 10a lighter and use less material during the manufacturing process. FIG. 5 shows an embodiment in which the openings 20b are a different number and arrangement as FIG. 4. In fact, any number or arrangement of openings may be used in various embodiments as deemed necessary. While FIGS. 4 and 5 show that the openings are circular, it will be understood, that the shapes of the openings 20a, 20b, are immaterial and they may be of any shape such as ovular, square, triangular, rectangular, irregular, or regular. The openings 20a, 20b, may also be arranged in grids or randomly.

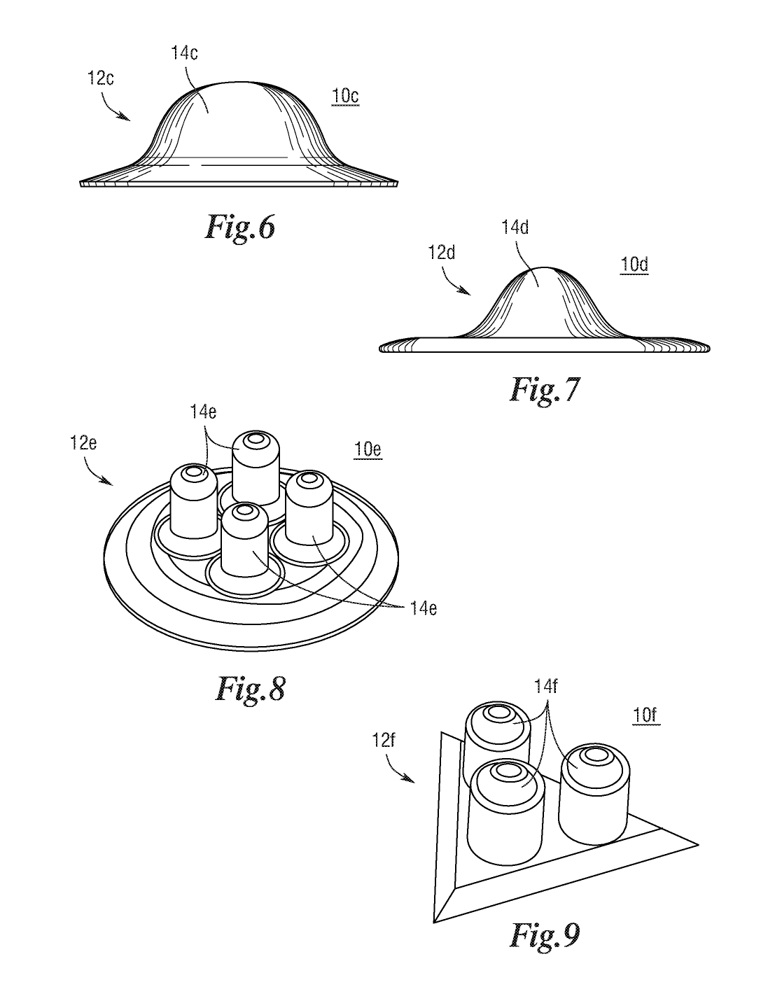

The dimensions of the working area may also be modified in different embodiments. While FIG. 1 shows the working area 14 having a sloped surface as shown. FIG. 6 shows an embodiment of trigger point treatment device 10c having a wider working area 14c than earlier shown embodiments. FIG. 6 shows an embodiment of trigger point treatment device 10c having a wider working area 14c than earlier shown embodiments. In fact, the working area of the trigger point treatment device could be any shape, such as conical, spheroidal, orbicular, ball shaped, or peaked.

The embodiments of trigger point treatment devices shown this far show working areas that comprise a single shape protruding towards the use. Other configurations of working areas are possible. For example, FIG. 8 shows an embodiment of trigger point treatment device 10e in which the working area 14e comprises multiple shapes, in this example four pillars, that protrude towards the user. It is also possible that these pillars may be detachable from the convex side 12e and replaced with pillars of other shapes, sizes, or stiffness as may be customizable for the embodiment. The working areas of the trigger point treatment device could comprise other accessories to augment the trigger point treatment device such as incorporating heat generation devices, electrical pulse generation devices, or vibration generation devices to the working area. These accessories could be used to supplement the self-applied pressure imposed by the user against the working area.

FIG. 9 shows that the overall shape of the trigger point treatment device 10f does not have to be circular as shown in earlier embodiments. In this case the trigger point treatment device 10f is triangular. Other shapes of the device are possible, such as square, rectangular, ovular, etc., so long as the convex side enables the trigger point treatment device 10f to be attached to a surface.

This invention has been described with reference to several preferred embodiments. Many modifications and alterations will occur to others upon reading and understanding the preceding specification. It is intended that the invention be construed as including all such alterations and modifications in so far as they come within the scope of the appended claims or the equivalents of these claims.

What is claimed is:

1. A trigger point treatment device comprising:
a convex side comprising a working area against which a user may present trigger point areas for the application of self-applied pressure; and
a concave side for mounting said trigger point treatment device to a surface by orienting said concave side against the surface and applying pressure against said convex side to create negative pressure between said concave side and the surface.

2. The trigger point treatment device of claim 1 in which said working area is used for any of the temporary relief of myofascial pain syndrome, massage therapy, minor pain relief, muscle soreness, and muscle fatigue.

3. The trigger point treatment device of claim 1 in which said working area further comprises one of a heat generation device, an electrical pulse generation device, or a vibration generation device.

4. The trigger point treatment device of claim 1 in which said working area comprises a single shape protruding towards the user.

5. The trigger point treatment device of claim 1 in which said working area comprises multiple shapes protruding towards the user.

6. The trigger point treatment device of claim 1 in which said working area has a shape that is one of conical, spheroidal, orbicular, ball shaped, or peaked.

7. The trigger point treatment device of claim 1 in which said working area is detachable from said convex side.

8. The trigger point treatment device of claim 1 that is made from plastic or rubber.
